(19) United States
(12) Patent Application Publication
(10) Pub. No.: US 2023/0301622 A1
(43) Pub. Date: Sep. 28, 2023

(54) DENTAL DEVICE AND METHODS

(71) Applicants: Tanmoy BHATTACHARJEE, Dunedin, Auckland (NZ); Paul David HARRIS, Stokes Valley Lower Hutt (NZ); Warwick John DUNCAN, Dunedin, Auckland (NZ)

(72) Inventors: Tanmoy BHATTACHARJEE, Dunedin, Auckland (NZ); Paul David HARRIS, Stokes Valley Lower Hutt (NZ); Warwick John DUNCAN, Dunedin, Auckland (NZ)

(73) Assignee: Otago Innovation Limited, North Dunedin, Auckland (NZ)

(21) Appl. No.: 18/041,512

(22) PCT Filed: Aug. 13, 2021
(86) PCT No.: PCT/IB2021/057458

§ 371 Date: Feb. 13, 2023

(30) Foreign Application Priority Data
Aug. 13, 2020 (NZ) 767112

(51) Int. Cl. A61B 8/08

(52) U.S. Cl. CPC A61B 8/0858; A61B 2562/0247; A61B 2562/0252

(57) ABSTRACT

A device for detecting properties of soft tissue. The device comprises a probe coupled to drive means to urge the probe against the soft tissue, a detector for determining applied stress to the soft tissue, and an ultrasonic transmitter configured to, in use, transmit ultrasound waves through at least a portion of the probe and through the soft tissue. A system including the device is also provided along with a corresponding method.

### FIELD OF INVENTION

The disclosure relates to a dental device and associated methods. More particularly, the disclosure relates to detecting properties of periodontal soft tissue and may have application to the detection of periodontitis.

### BACKGROUND

The goal of diagnostic procedures for periodontitis is the earliest possible identification of the disease process as this allows the most effective preventive procedures and least invasive interventions. Examination techniques for periodontal disease have not changed in a century and involve painful manual probing of the periodontal soft tissues (gums) around teeth. One prior art probe developed in the 1930's is a stainless steel, blunt-tipped hand instrument with a tip 13 mm long with a 1 mm diameter. This is still the most commonly-used probe in dental practice. However, other modified probes have been developed including (1) pressure-sensitive constant pressure probes, (2) constant pressure automated probes, (3) so-called 3-D probes which are still under development, and (4) 3D non-invasive probes using ultrasonography or other imaging modalities. However, developments beyond the first generation of probes offer few advantages and their use remains mainly confined to research.

The modified probes have offered little improvement to probing procedure and, as a consequence, the measurement error of electronic probes is not substantially less than that of manual probes. Further, there have been concerns around patient discomfort when using modified probes.

Accuracy of periodontal pocket measurements is impacted by (1) the disease process, local anatomy, tissue inflammation and loss of elasticity, and pain on probing, (2) probe type, shape, and size, and (3) operator technique, including angulation and probe force. Probing generally returns good inter-examiner agreement in healthy, non-inflamed tissues but becomes much less reliable in the presence of inflammation which is characterised by ulceration of the epithelial attachment at the bottom of the pocket, and loss of the supporting connective tissues underlying this. The pressure applied during probing is a major variable that is difficult to control and for this reason, although probing pocket depth is still the major diagnostic criteria used to determine the presence of existing disease, its accuracy has been questioned and it is not considered an accurate predictor for progression of disease.

WO2019008586 describes an intra oral scanner (IOS) having a probe for detecting elasticity of the gum or periodontal soft tissue. The probe applies force to the tissue and a force sensor mounted on the tip of the probe is used to detect the force applied by the probe. The scanner detects the resultant deformation of the tissue and the force and deformation are used to determine the elasticity or softness of the tissue.

Referring to FIG. 3D of WO2019008586, the imager 306 is used to determine displacement. However, the ball at the tip of the probe obstructs the view of the imager. At best, only the uppermost edge of the contact (or the contact closest the imager) could be observed by the imager. Consequently, it is unclear how the scanner can measure displacement, at least accurately. It is also unclear as to how the surface area of contact between the ball and the tissue is determined, which is critical to determining stress. If the displacement or the area of contact cannot be determined, then the strain or stress respectively cannot be determined. To determine elasticity, one must determine stress and strain, where the strain is equal to the deformation divided by the tissue thickness. So one also needs to measure the thickness of the tissue. There is no indication in WO2019008586 that this is determined, how it would be determined or why it would be determined.

It is an object to provide apparatus and/or methods for detecting properties of periodontal soft tissue that overcome or ameliorate at least one of the drawbacks of WO2019008586, or at least provides a useful alternative over this or other prior art approaches.

### SUMMARY OF THE INVENTION

According to a first aspect, there is provided a device for detecting properties of soft tissue, the device comprising a probe coupled to drive means to urge the probe against the soft tissue, a detector for determining applied stress to the soft tissue, and an ultrasonic transmitter configured to, in use, transmit ultrasound waves through at least a portion of the probe and through the soft tissue.

The probe may comprise a shaft or other substantially rigid body.

The probe and the drive means may be configured to move the probe (e.g. linearly) in a reciprocating motion towards and away from the soft tissue.

The drive means may comprise a motor.

The detector may comprise a load cell.

The probe may include the detector.

The device may comprise an ultrasonic receiver to receive the ultrasound waves generated by the ultrasonic transmitter, after passing through the soft tissue.

The device may comprise an ultrasonic transducer. The ultrasonic transducer may convert electrical signals from the transmitter into ultrasound pulses and/or convert received ultrasound waves into electrical signals and provide these to the receiver.

The probe may comprise the ultrasonic transducer.

The device may comprise an ultrasonic delay line provided at or proximate to a patient-engaging tip of the probe.

The ultrasonic delay line may be provided to a patient-engaging side of the ultrasonic transducer.

The device may be configured to detect properties of periodontal soft tissue and/or to have application to the detection of periodontitis.

The device may comprise biasing means to provide a biasing force for the probe towards the soft tissue, said biasing force being additional to the force generated by said drive means.

The drive means may be configured to move a soft tissue engaging tip of the probe towards and away from the soft tissue over one or more cycles.

According to a second aspect, there is provided a system for detecting properties of soft tissue, the system comprising the device of the first aspect, and a processor configured to:

generate signals to drive the motor to apply a varying force by the probe to the soft tissue and to transmit ultrasound waves through the soft tissue,

determine an applied stress, and

determine a thickness of the soft tissue.

The processor may be configured to determine variations in the thickness of the soft tissue as a result of the varying force applied to the soft tissue.

According to a third aspect, there is provided a method of detecting properties of soft tissue, comprising applying a varying force to the soft tissue, transmitting ultrasound waves through the soft tissue, determining an applied stress, and determining a thickness of the soft tissue.

The method may comprise determining variations in the thickness of the soft tissue as a result of the varying force applied to the soft tissue.

The step of applying a varying force may comprise moving a probe or at least a portion of a probe towards and away from the soft tissue.

The step of transmitting may comprise transmitting said ultrasound waves through an ultrasonic delay line.

According to a fourth aspect, there is provided a device for determining the thickness of tissue. The device of this aspect may comprise some or all or be integrated with the device of the first aspect. More particularly, the device of the first aspect may be configured to provide the functionality of the device of the fourth aspect and vice versa.

Thus, the device of the fourth aspect may comprise a probe for contacting a portion of tissue, an ultrasonic transmitter configured to, in use, transmit ultrasound waves through at least a portion of the probe and through the soft tissue, and an ultrasonic receiver to receive the ultrasound waves generated by the ultrasonic transmitter, after passing through the tissue.

The device may comprise an ultrasonic transducer. The ultrasonic transducer may convert electrical signals from the transmitter into ultrasound pulses and/or convert received ultrasound waves into electrical signals and provide these to the receiver.

The probe may comprise the ultrasonic transducer.

The device may comprise an ultrasonic delay line provided at or proximate to a patient-engaging tip of the probe.

The ultrasonic delay line may be provided to a patient-engaging side of the ultrasonic transducer.

The probe may be configured to controllably apply a varying force to the tissue. To this end, the device of the fourth aspect may, for example, comprise the drive means, load cell and detector of the device of the first aspect.

More particularly, according to preferred embodiments, the device is configured to determine tissue thickness and its variation while varying an applied stress during palpation. According to preferred embodiments, the device is configured to determine tissue thickness whilst varying applied stress of the probe against the tissue. A controller may be provided to determine the tissue thickness from stress and strain data gathered during palpation, details of which are provided with respect to the device of the first aspect. Additionally or alternatively, one or more of the palpation stress, pre-load stress may be varied while measuring tissue thickness to improve measurement accuracy. The controller may be configured to use this data to determine the tissue thickness at or near a relaxed state (i.e. little or no applied stress) by extrapolation.

The controller may be integrated with the device or may be in part or wholly located external to the device with a communicative coupling provided to a remote controller.

One or more further features of the device of the first aspect may be incorporated in the device of the second aspect. Corresponding methods are also disclosed.

### BRIEF DESCRIPTION OF THE DRAWINGS

These and other features, aspects, and advantages of the present disclosure will be described with respect to the following figures, which are intended to illustrate and not limit the preferred embodiments.

FIG. 1 is a schematic sectional view of a device according to one embodiment.

FIG. 2 is an example prototype device according to one embodiment.

FIG. 3 is a schematic circuit diagram of a device according to one embodiment.

FIGS. 4 to 9 provide sample results obtained using the prototype shown in FIG. 2.

FIG. 10 is a chart showing moving averages of elastic modulus determined using a prototype according to an embodiment of the invention for every palpation cycle recorded from patient gingival tissue.

FIG. 11 is a chart showing moving averages of elastic modulus determined using a prototype according to an embodiment of the invention with gingival inflammation status indicated as derived from patient sample histology.

FIG. 12 is a recreated image of a probe according to an embodiment of the invention measuring cadaver gingiva.

FIG. 13 is a high resolution image of a stained histology specimen with the measurement location identified by “X” and the thickness as marked.

FIG. 14 provides sample results including display of gingival thickness.

FIG. 15 is a correlation plot between gingival thicknesses determined using a probe according to an embodiment of the invention and histology-based measurements.

### DETAILED DESCRIPTION

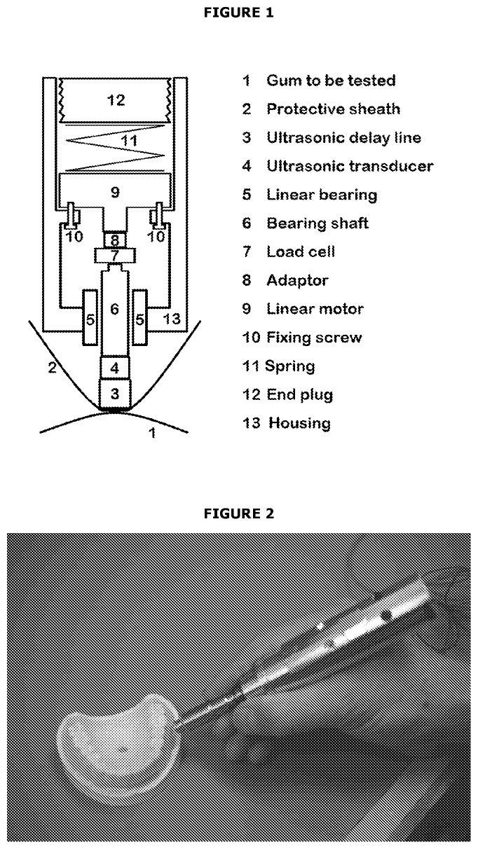

FIG. 1 is a schematic sectional view of a device or probe according to one embodiment of the disclosure, with the tip thereof abutting tissue to be tested, such as gum 1. A protective sheath 2 may be provided about the patient-engaging part of the apparatus to prevent liquids and other material getting into the interior of the device and also to enable the device to be sterilised more easily between patients. To this end, the sheath 2 may be replaceable and/or washable.

An ultrasonic delay line 3 (e.g. a block of material whose function is to provide an ultrasonic delay to separate transmitted and received signals at the transducer) is provided at the tip of the device on the non-patient engaging side of the sheath 2. The ultrasonic delay line 3 is coupled to an ultrasonic transducer 4 which is mounted to a shaft 6 provided within bearing housing 5. The shaft 6 and bearing housing 5 allow for the patient engaging tip of the device to move towards and away from the gum 1.

The end of the shaft 6 farthest from the gum 1 is coupled to a load cell 7 which in turn is coupled to an adapter 8. The adapter 8 couples the load cell 7 to linear motor 9 which generates the desired linear movement of the shaft 6 within the bearing housing 5. The body or housing of the linear motor 9 is fixedly coupled to the housing 13 of the device, for example, using screws 10. According to some embodiments, the coupling is releasable and it is possible to remove the motor 9 e.g. for servicing or replacing. Other parts of the device may also be removable and/or replaceable. The bearing housing 5 is also fixedly coupled to the housing 13 to fix the bearing housing 5 spatially relative to the motor 9, such that the linear movement is limited to parts of the device on the patient-side of the motor 9.

The housing 13 is closed off at the non-patient engaging end. For example, a plug 12 may be provided. A spring 11 or restoring means may be provided between the motor 9 and the plug 12 or other closure to provide a steady gum loading, acting in addition to a cyclic palpation loading provided by the linear motor. In other embodiments the linear motor may provide both steady and palpation loading functions.

While not shown, the device preferably includes a controller for controlling operation of the device and/or is communicatively couplable to a remotely positioned controller. For example, control of the device to enable tests to be conducted may be performed by a local controller inside the housing 13 but post-processing of results may be performed using external equipment wired or wirelessly communicatively coupled to the device.

Further, the device may include an internal power supply and/or include a connector for connecting to an external power supply.

FIG. 2 is an example prototype device according to one embodiment. This prototype is in accordance with the arrangement shown in FIG. 1.

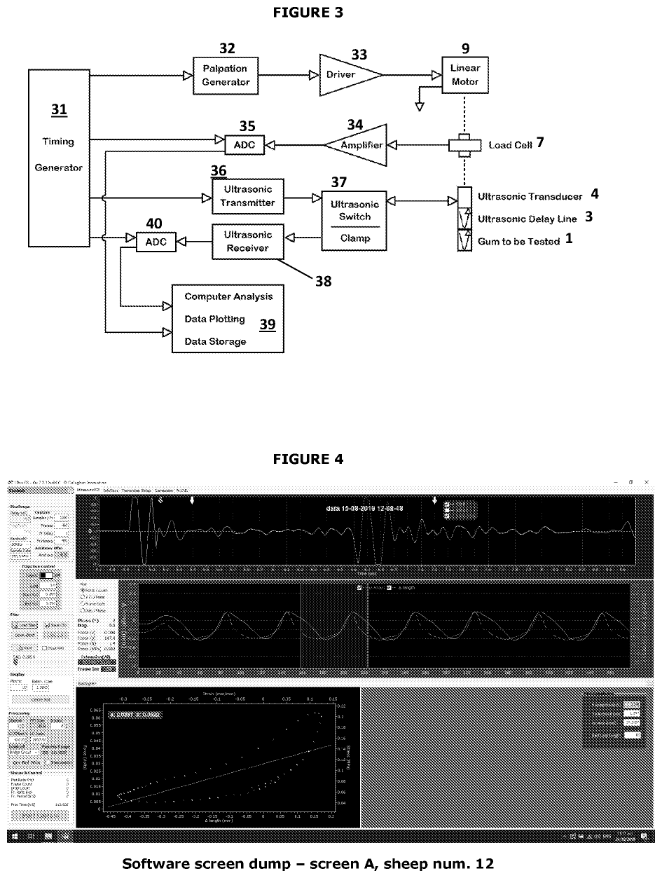

FIG. 3 is a schematic circuit diagram of a device according to one embodiment. Elements common to FIGS. 1 and 3 have been identified using the same reference numerals.

Central to the circuit is timing generator 31. The palpation generator 32 receives a signal from the timing generator 31 and outputs a signal that is amplified by driver 33 and applied to linear motor 9 to impart a stress on the tissue to be tested, e.g. gum 1.

Instantaneous applied stress is computed by measuring a voltage derived from the load cell 7. The load cell 7 is in a bridge configuration and generates a signal that is amplified by amplifier 34 and provided to processor 39 via analog to digital converter 35. The force and stress on the gum are then determined. This is readily possible due to the defined profile (namely, a known surface area) of the patient-engaging tip of the device. The tip preferably has a small enough area that the entire end wall of the tip engages the tissue in use but is not so small as to be likely to injure the patient by damaging the tissue. The tip should broadly fall in the range 0.5 mm diameter to 6 mm diameter, or be of similar area if non-circular. The degree to which the gum properties shear and anisotropy in material stiffness (or whatever term you use) could influence measured data will also be related to the tip diameter, varying with diameter. The profile of the tip is also important so as to avoid ambiguity in the contact area. Thus, as shown in FIGS. 1 and 2, the tip may be defined by a flat, planar surface (or substantially flat and planar) with the probe body extending orthogonally, or substantially orthogonally, away from the planar surface thereof.

Instantaneous strain is measured ultrasonically. Pulse-echo measurements record reverberation signals including of the acoustic delay line 3 and the extent of the gum beyond (i.e. the thickness of the gum at the point being tested) and from this strain is computed. The strain computation does not necessitate knowledge of an acoustic velocity. Strain data for measurements over a palpation cycle, or cycles, will exhibit strain terms that relate to the palpation stress cycles at the constant preload stress. Palpation is preferably at 5 Hz to 150 Hz. From the varying strain associated with the palpation cycle, or cycles, and its causal varying stress the elasticity of the gum tissue can be characterised; varying the preload stress allows a wider range of viscoelastic properties to be characterised. Where a transducer array is used the variation of strain over the measured area is able to be determined.

It is clinically desired to have knowledge of tissue thickness when the tissue is in a relaxed or near relaxed state i.e. little or no applied stress. The pulse-echo measurement, for a given acoustic velocity, provides tissue thickness and its variation with varying applied stress during palpation. The device can be configured to indicate the relaxed tissue thickness from the strain and stress data gathered during palpation, or by varying the palpation stress, varying the preload stress, or a combination of these for an improved tissue thickness measurement.

To improve accuracy, computation of tissue properties may use measurements over more than one palpation cycle. 8 cycles are currently preferred as providing the desired accuracy, providing indication of accumulative gum displacements (thinning) over several palpation cycles should this occur, and being a short measurement time for patient scanning.

Referring again to FIG. 3, an ultrasonic transmitter circuit 36 is also coupled to the timing generator 31 and when signalled by the timing generator 31, generates an electrical signal to stimulate the ultrasonic transducer 4 via ultrasonic switch/clamp 37. The transducer 4 converts this electrical signal to an ultrasound wave. The ultrasound wave is transmitted through the ultrasound delay line 3 and gum to be tested and echoes are returned to the ultrasonic transducer 4 via the ultrasound delay line 3, these ultrasound signals are converted to electrical signals by the ultrasonic transducer 4 and via the ultrasonic switch/clamp 37 passed to the ultrasonic receiver circuit 38. The resultant electrical signal may pass through another analog to digital converter 40 before being provided to processor 39 for analysis. Other embodiments use alternative means to generate such a pulse-echo system, whilst others may use separate transmitter and receiver transducer arrangements.

As indicated previously, processor 39 may be external to the device and may include a display and memory.

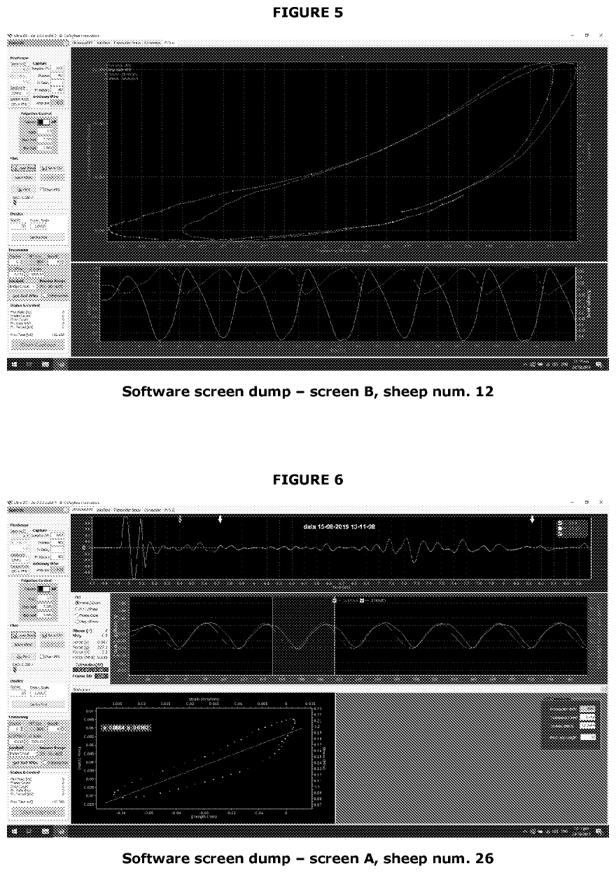

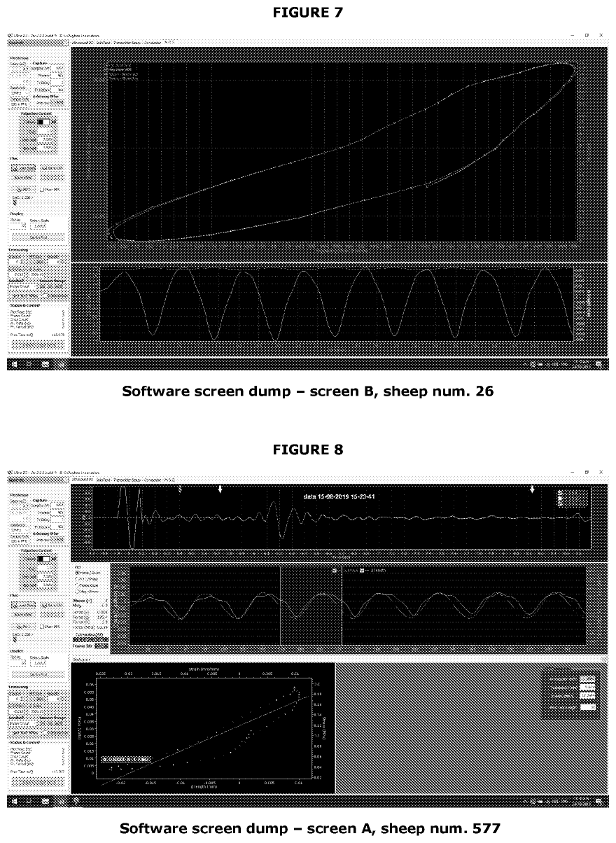

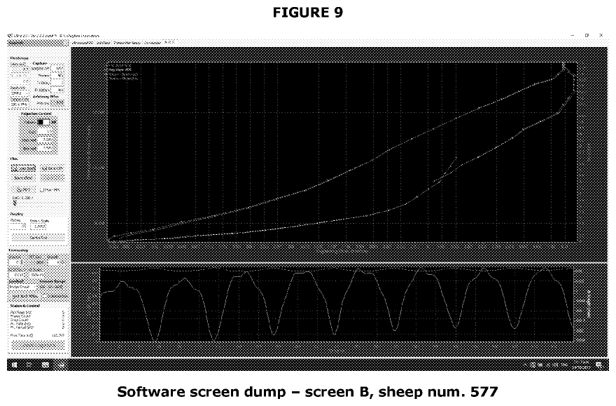

The data or results may be provided graphically. FIGS. 4 to 9 show example results for the gums of three sheep. There are two plots, A and B, for each sheep with the A plot (FIGS. 4, 6 and 8) being the results generated at runtime and the B plot (FIGS. 5, 7 and 9) generated subsequently from the data. FIGS. 5, 7 and 9 correspond to the results from FIGS. 4, 6 and 8, respectively. Accordingly FIGS. 4 and 5 present results from the same core data taken from the same probing of one particular sheep. The same applies to FIGS. 6 and 7 and FIGS. 8 and 9 with each pairing of Figures relating to a different sheep.

Referring to FIG. 4, the uppermost plot or graph shows the ultrasound pulse-echo trace(s). The large increases in amplitude towards the left hand side of the plot correspond to the echo from the tip of the delay line 3. The large increases in amplitude towards the middle of the plot correspond to the echo from the far side of the gum i.e. the gum-tooth/bone interface. Thus a thickness of the gum 1 can be determined and strain can be calculated.

The middle plot shows directly the influence of palpation and palpation cycles. Whilst other options exist e.g. the measured ADC voltages, here the data for delta length (mm) and load cell force (grams) is being displayed with time during the palpation cycle burst. The delta length is the change in thickness of the tissue during the palpation cycles i.e. how much the tissue compresses and expands during the variations in the force. The plot shows 8 cycles.

The bottom plot shows the same data on an XY plot. This is a surrogate plot for elasticity and provides an immediate indication for the form of the data. It is not an exact stress-strain curve since it provides the immediate data using user-entered parameters for tissue thickness (to relate to strain) and area (to relate to stress), however the data provides an immediate indication for disease assessment.

The plots shown in FIG. 5 are produced currently retrospectively though in some embodiments may be generated concurrently. Consequently, the dataset can be used as a whole so that, for instance, tissue thickness can be determined over the palpation cycles and burst. The upper plot shows the resultant more accurate stress-strain curve and the lower plot shows the tissue thickness (length) and change in thickness (delta length) during the palpation cycle burst.

As indicated previously, the plots of FIGS. 6 to 9 correspond to those of FIGS. 4 and 5 but for different sheep.

Clinical data suggests gum disease quickly depletes collagen structures within the tissues, significantly reducing the tissue stiffness (modulus of elasticity). Tissue stiffness is indicated by the slope of the elasticity plot in FIG. 4 (bottom plot) and/or stress-strain curve (FIG. 5, top plot).

There is the added clinical observation that gum disease leads to swelling and excess fluids within the tissues, this excess fluid being “extruded” by the preload and palpation for an apparent downwards drift in tissue thickness (thinning) of the tissues over several palpation cycles—this drift is most easily observed in the middle plot of FIG. 4.

Ultrasonic backscatter, reflections and scattering from small structures in the acoustic path (e.g. collagen structures), magnitude is related to the density of scattering structures in the acoustic path. Disease depletion of the collagen structures of the gums will diminish the backscatter signal of the gum tissue throughout its bulk relative to that for healthy tissue.

As the backscatter signal provides a measure of collagen content it may also be used in the strain computation to account for subtle localised variation in the tissue ultrasonic signal speed associated with varying collagen content.

The magnitude of signals reflected from interfaces with the gum or by structures within the gum, and reverberations of these, may vary with tissue health and be a factor used in reporting tissue health.

The plots 5, 7 and 9 show varying degrees of elastic nonlinearity i.e. deviations from a purely (linear) elastic response, and it is considered this is related to the substructures within the gum and the gum response to the applied stress. At low stress and low collagen density/packing one may expect the gum to exhibit a low elastic modulus, as the stress is increased, a sufficiently high stress may be applied so that the collagen density/packing appears dense to the extent that the collagen structures collectively makes the gum tissue exhibit a high modulus. Alternatively, high collagen density/packing is expected to exhibit high elastic modulus at low stress levels. The degree of nonlinearity and levels of stress at inflection points is thought to relate to collagen density/packing and hence to gum health.

The palpation frequency, and relative magnitude of the palpation to the steady state preload, is thought to have a bearing on the degree to which fluids within the gum tissues, in particular excess fluids related to swelling and intracellular fluids, are able to flow within the gum. At high palpation frequencies the fluid may effectively be trapped by its viscosity within the gum tissues and unable to flow in response to the cyclic palpation stress and exhibit a high (visco)elastic response. At progressively lower frequencies, including a steady state preload, flow is expected to increase and this will be reflected in an apparent low (visco)elastic response. The frequency for inflections in the relationship may relate to gum health.

### Testing

Testing of human patients was also undertaken.

Patients with either healthy gums or with gum disease, who had been referred for extraction of one or more teeth at the School of Dentistry of the University of Otago, were recruited.

Inclusion criteria: Participants were required to be aged 18 years or over and have one or more teeth deemed unable to be restored and requiring extraction, or requiring gum surgery with removal of soft tissue, as determined by their primary health care provider.

Exclusion criteria: Impacted wisdom teeth, teeth with acute inflammation (large and painful abscesses), and patients with significant systemic disease, bleeding diatheses or taking anticoagulant medications requiring complex pre- and post-surgical management were excluded. Pregnant subjects were also excluded.

The Principal Investigator (PI) screened potential participants for evidence of periodontal disease. He explained the study to them, informed them of possible risks, and provided them with further reading material to decide whether they would like to participate. Signed consent forms were obtained from those who agreed to be part of the study.

### Clinical Recordings, Surgical Treatment, and Questionnaire

The patients were prepared for periodontal (gum) surgery and/or dental extraction as per routine dental practices. Clinical recordings were obtained prior to surgery. Standard periodontal recordings by manual probing with a periodontal probe were recorded and dental radiographs were taken of the teeth requiring surgery. The PI then obtained ultrasound recordings of the same tooth, by touching the tip of the prototype device of the present invention, covered by a latex finger cot, to the gingiva (gum) overlying the identified tooth or teeth. Medical-grade glycerol was applied external to the finger cot to act as an ultrasound coupling medium. Three to five readings were taken, each requiring approximately 10 seconds. The collected data were saved by renaming them with patient file numbers. Participants were then given a brief questionnaire about their experience of the ultrasound recording process. The teeth were then anaesthetised with standard dental local anaesthesia and surgery completed as per routine treatment protocols. A small (5×5 mm) amount of gingiva (gum) was biopsied at the same time for microscopic examination. The sites were sutured closed following surgery.

### Histological Analysis

All histopathological studies were conducted in the Oral Pathology lab at the Faculty of Dentistry of the University of Otago. Gingival biopsies were formalin-fixed, paraffin-embedded, sectioned and stained with haematoxylin and eosin for histological examination. Experienced pathologists categorized the histological images as healthy, lightly inflamed, or inflamed.

### Analysis of Data Gathered Using the Prototype

The data was analysed using the software used to analyse the sheep data previously discussed. This provided the elastic modulus per palpation cycle, plotted on a graph. A moving average was derived from the data points, one moving average line corresponding to each patient. These lines were compared with the histology-based assessments to assess correlation between the results of the two modalities.

### Results

### Participant Recruitment

Ten patients matched the inclusion and exclusion criteria and consented to be part of the study. One healthy participant also consented and was included in the study (tested using the prototype according to the invention only, without histology).

### Clinical Recordings, Surgical Treatment, and Questionnaire

Clinical recordings, measurements derived using the prototype, questionnaires (Table 1), and surgical treatment with biopsy was successfully completed for all ten patients. With regards to the questionnaires, 8 out of 10 patients reported that they did not feel any pain during the recordings taken using the prototype while one patient reported pain at an inflamed site, but not on a healthy site. Nine out of 10 patients said they would not mind if the test is performed every time they visited their dentist.

TABLE 1
Answers to questionnaires to patients after recordings were
completed using the prototype according to the invention

| | Did you feel any pain | Would you mind if | |
|---|---|---|---|
| | or discomfort when | the test is performed | |
| | the UltraD3 test | every time you visit | |
| Patient | was performed? | your dentist? | Comments |
| P1 | Yes | No | |
| P2 | No | No | Improve device |
| | | | aesthetics |
| P4 | Yes (inflamed side)/ | Yes | |
| | No (healthy side) | | |
| P5 | Yes | No | Develop narrative |
| | | | about the device |
| | | | to tell the patient |
| | | | while waiting for |
| | | | the test |
| P6 | No | No | Less vibration |
| P7 | No | No | |
| P8 | No | No | |
| P10 | No | No | |
| P11 | No | No | |
| P12 | No | No | |

### Histological Analysis

Of the 10 patients, one participant was categorized as healthy, four as lightly inflamed, and four as inflamed (Table 2).

TABLE 2
Histology-based inflammation status of tissue extracted
from gingival tissues of patients at the site
of recordings taken using the prototype

| Patient | Histology |
|---|---|
| T | Healthy |
| P1 | Inflamed |
| P2 | Lightly inflamed |
| P4 | Inflamed |
| P5 | Lightly inflamed |
| P6 | Abscess/Induration |
| P7 | Inflamed |
| P8 27 | Inflamed |
| P10 | Lightly inflamed |
| P11 | Healthy |
| P12 | Lightly inflamed |

### Analysis of Data Derived Using the Prototype

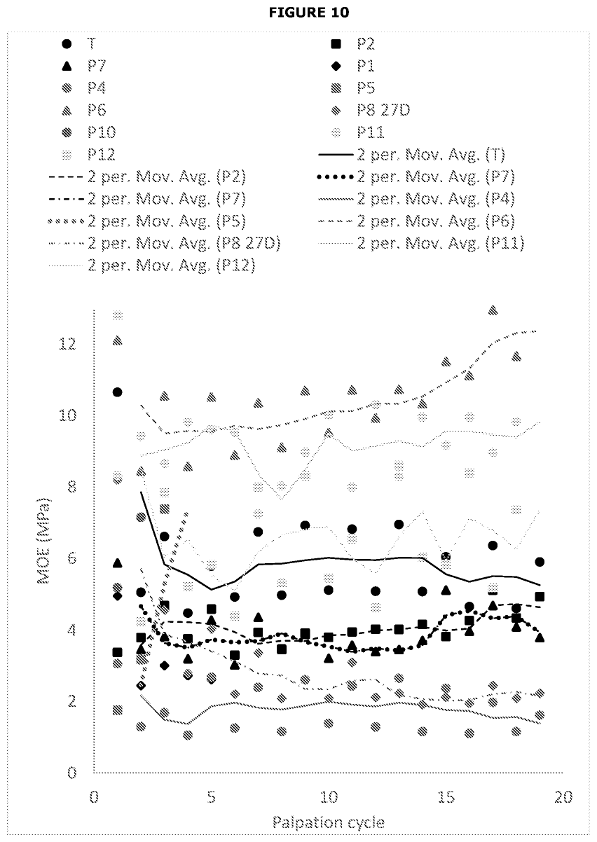

The elastic modulus could be retrieved from all 19 palpation cycles for 7 out of 10 patients (FIG. 10). For three participants (P5, P10, and P1), elastic modulus was obtained from only 2, 2, or 5 palpation cycles, respectively. Note, “T” represents the healthy volunteer.

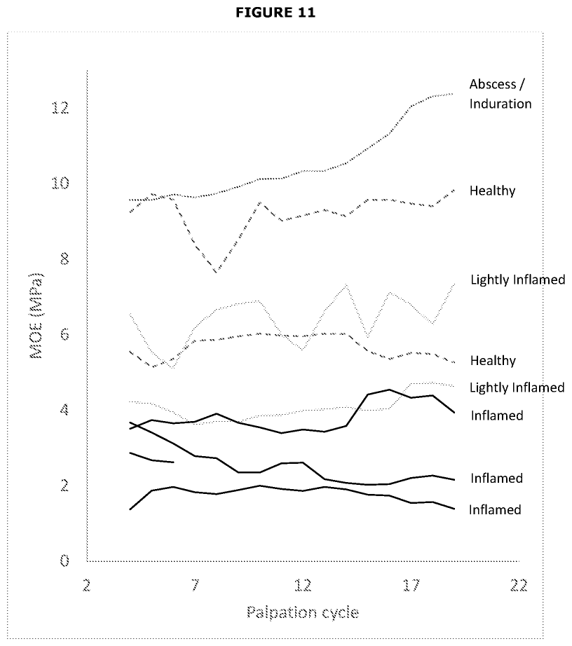

The elastic modulus moving average per patient with the result of the histology-based assessment provided to the right thereof is shown in FIG. 11.

### Discussion

There is a correlation between the elastic modulus analysis performed using the prototype of the invention and disease inflammation status; with healthy, lightly inflamed, and inflamed gingival tissue showing an elastic modulus ranging between 5-10 MPa, 4-7 MPa, and 1-4 MPa, respectively. The data from P5, which was histologically demonstrated as an acutely-inflamed abscess, was obtained from the border of the abscess (indurated tissue), which has been reported to have a higher elastic modulus. The study shows that the invention has potential for early diagnosis of periodontal disease.

### Further Analysis

Testing was performed to evaluate the accuracy of the prototype device in measuring gingival thickness compared to histology.

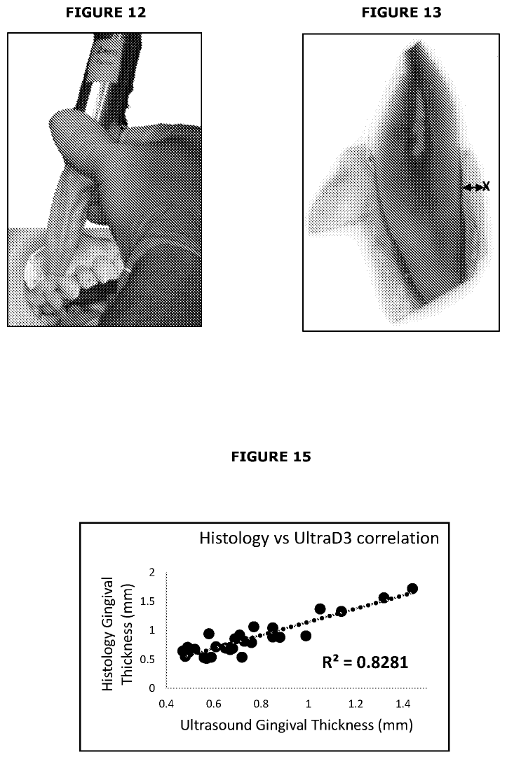

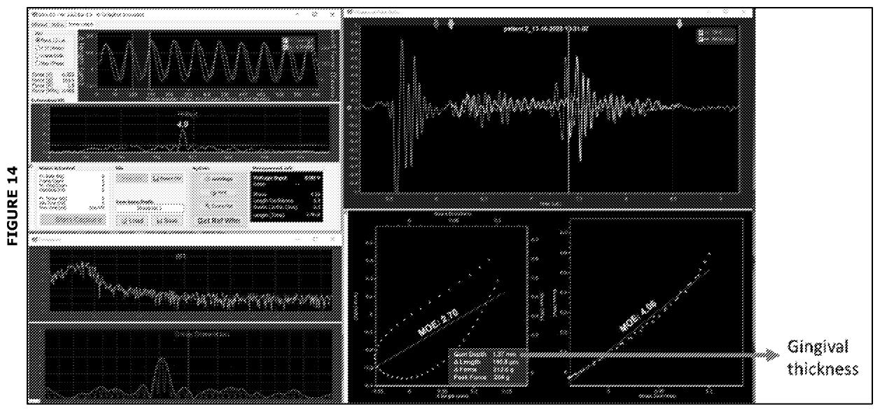

Methodology: The study used 27 gingiva from 8 Crosado-embalmed cadavers. The embalming technique was chosen as it ensures retention of tissue softness/pliability. Readings were acquired using the prototype from intact gingiva in situ (FIG. 12), followed by extraction of gingiva along with the teeth. The samples were resin embedded, sectioned, stained, and scanned at high resolution (2400 dpi). Gingival thickness was then measured from the images in ImageJ software (FIG. 13) using the 2400 dpi scanned image of a ruler to set scale. Measures for gingival thickness using the prototype were obtained directly using the software discussed above with resultant data shown in FIG. 14. A correlation plot was generated to compare gingival thickness measured by the two modalities.

Results: Table 3 lists the gingival thickness for different cadaver samples as measured using the prototype and Histology.

TABLE 3
Comparison of measurements of gingival thickness
derived using the prototype and histology

| | |
|---|---|
| Cadaver | Gum Depth (mm) |

| Sr. no. | Sample ID. | UltraD3 | Histology |
|---|---|---|---|
| 1 | 1045 26 | 0.99 | 0.906 |
| 2 | 1045 11 | 0.69 | 0.854 |
| 3 | 1028 31 | 0.58 | 0.942 |
| 4 | 1045 42 | 1.05 | 1.369 |
| 5 | 1057 24b | 0.77 | 1.062 |
| 6 | 1028 24p | 1.44 | 1.72 |
| 7 | 1045 24p | 1.32 | 1.561 |
| 8 | 1057 31 | 0.49 | 0.707 |
| 9 | 995 33 | 0.71 | 0.918 |
| 10 | 1028 11 | 0.85 | 1.044 |
| 11 | 1028 26 | 1.14 | 1.327 |
| 12 | 1057 33 | 0.47 | 0.643 |
| 13 | 998 33 | 0.51 | 0.665 |
| 14 | 1045 31 | 0.52 | 0.675 |
| 15 | 1028 43 | 0.61 | 0.717 |
| 16 | 998 11 | 0.73 | 0.812 |
| 17 | 1028 33 | 0.48 | 0.552 |
| 18 | 1045 33 | 0.65 | 0.696 |
| 19 | 1017 11 | 0.85 | 0.886 |
| 20 | 1040 11 | 0.76 | 0.791 |
| 21 | 920 43 | 0.68 | 0.69 |
| 22 | 1057 26 | 0.88 | 0.881 |
| 23 | 1045 24b | 0.67 | 0.669 |
| 24 | 1057 42 | 0.56 | 0.531 |
| 25 | 1017 31 | 0.59 | 0.538 |
| 26 | 998 31 | 0.57 | 0.517 |
| 27 | 1017 33 | 0.72 | 0.538 |

The correlation plot (FIG. 15) shows a correlation coefficient (R2) of 0.83, which suggests a good agreement between the two sets of gingival thickness measurements.

In the testing conducted, the prototype was configured by compressing the gingiva, which results in slightly underestimating gingival thickness. However, the system can be configured by appropriate programming of the software to reduce the applied pressure while performing thickness measurement, and project the data point at near zero pressure, providing more accurate measure of the thickness.

Importance: Gingival thickness measurement plays a vital role in periodontic, orthodontic, and aesthetic surgeries. In addition to diagnosing/detecting periodontal disease early, embodiments of the invention can also become a vital tool for the above-mentioned surgeries by providing gingival thickness measurements.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

Reference to any prior art in this specification is not, and should not be taken as, an acknowledgement or any form of suggestion that that prior art forms part of the common general knowledge in the field of endeavour in any country in the world.

It should be emphasized that many variations and modifications may be made to the embodiments described herein, the elements of which are to be understood as being among other acceptable examples. All such modifications and variations are intended to be included herein within the scope of this disclosure and protected by the following claims. Further, nothing in the foregoing disclosure is intended to imply that any particular component, characteristic or process step is necessary or essential.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein.

1. A device for detecting properties of soft tissue, the device comprising:
a probe coupled to drive means to urge the probe against the soft tissue;
a detector for determining applied stress to the soft tissue; and
an ultrasonic transmitter configured to, in use, transmit ultrasound waves through at least a portion of the probe and through the soft tissue.

2. The device of claim 1, wherein the probe comprises a shaft.

3. The device of claim 2, wherein the probe and the drive means are configured to move the probe in a reciprocating motion towards and away from the soft tissue.

4. The device of claim 1, wherein the drive means comprises a motor.

5. The device of claim 1, wherein the detector comprises a load cell.

6. The device of claim 1, wherein the probe includes the detector.

7. The device of claim 1, comprising an ultrasonic receiver to receive the ultrasound waves generated by the ultrasonic transmitter.

8. The device of claim 1, comprising an ultrasonic transducer.

9. The device of claim 7, wherein the probe comprises the ultrasonic transducer.

10. The device of claim 1, comprising an ultrasonic delay line provided at or proximate to a patient-engaging tip of the probe.

11. The device of claim 10, wherein the ultrasonic delay line is provided to a patient-engaging side of the ultrasonic transducer.

12. The device of claim 7, configured to determine a thickness of the tissue.

13. The device of claim 12, wherein the drive means is configured to controllably apply a varying force to the tissue during said determination.

14. The device of claim 1, configured to detect properties of periodontal soft tissue and/or to have application to the detection of periodontitis.

15. The device of claim 1, comprising biasing means to provide a biasing force for the probe towards the soft tissue, said biasing force being additional to the force generated by said drive means.

16. The device of claim 1, wherein the drive means is configured to move a soft tissue engaging tip of the probe towards and away from the soft tissue over one or more cycles.

17. A system for detecting properties of soft tissue, the system comprising:
the device of claim 1; and
a processor configured to:
drive the motor to apply a varying force by the probe to the soft tissue;
transmit ultrasound waves through the soft tissue;
determine an applied stress; and
determine a thickness of the soft tissue.

18. The system of claim 17, wherein the processor is configured to determine variations in the thickness of the soft tissue as a result of the varying force applied to the soft tissue.

19. A method of detecting properties of soft tissue, comprising:
applying a varying force to the soft tissue;
transmitting ultrasound waves through the soft tissue;
determining an applied stress; and
determining a thickness of the soft tissue.

20. The method of claim 19, comprising determining variations in the thickness of the soft tissue as a result of the varying force applied to the soft tissue.

21. The method of claim 19, wherein the applying a varying force comprises moving a probe towards and away from the soft tissue.

22. The method of claim 19, wherein said transmitting comprises transmitting said ultrasound waves through an ultrasonic delay line.
